(19) United States
(12) Patent Application Publication
(10) Pub. No.: US 2023/0079706 A1
(43) Pub. Date: Mar. 16, 2023

(54) Solid Cleansing Compositions and Methods for the Same

(71) Applicant: Colgate-Palmolive Company, New York, NY (US)

(72) Inventors: Jesus Ivan ROMERO, Ciudad de México (MX); Aaron COHEN, New Brunswick, NJ (US); Sara MORALES, Ciudad de México (MX); Vinay BHARDWAJ, Piscataway, NJ (US)

(73) Assignee: Colgate-Palmolive Company, New York, NY (US)

(21) Appl. No.: 17/943,103

(22) Filed: Sep. 12, 2022

Related U.S. Application Data
(60) Provisional application No. 63/243,848, filed on Sep. 14, 2021.

(51) Int. Cl. A61K 8/49; A61K 8/11; A61K 8/34; A61K 8/02; A61Q 19/02; A61Q 19/10

(52) U.S. Cl. CPC A61K 8/498; A61K 8/11; A61K 8/347; A61K 8/0216; A61Q 19/02; A61Q 19/10

(57) ABSTRACT

Solid cleansing compositions and methods for treating, decreasing, reducing, or preventing hyperpigmentation of skin, and/or methods for regulating melanin in or on skin are provided. The solid cleansing composition may include a delivery vehicle and one or more whitening agents. The whitening agents may include one or more of kojic acid, phenylethyl resorcinol, or combinations thereof. The whitening agents may be configured to regulate melanin in or on skin, and the delivery vehicle may be configured to stabilize the one or more whitening agents.

This application claims the benefit of U.S. Provisional Patent Application No. 63/243,848, filed on Sep. 14, 2021, which is incorporated herein by reference to the extent consistent with the present disclosure.

### BACKGROUND

Consumers are often concerned with the characteristics of their skin, including the degree of pigmentation, freckles, darkening caused by exposure to sunlight, and/or age spots. The characteristics of skin are often directly or indirectly related to melanin, a pigment synthesized by melanocytes that gives skin color. Additionally, some skin disorders are often associated with the overproduction and/or irregular distribution of melanin on skin (e.g., hyperpigmentation).

In view of the foregoing, consumers often utilize personal care compositions including one or more skin lightening agents to lighten skin, and/or treat, prevent, or inhibit skin disorders related to the production of melanin. For example, consumers often utilize personal care compositions incorporating skin lightening agents, such as Kojic acid. Kojic acid is a natural metabolite produced by fungi that has the ability to inhibit tyrosinase activity in the synthesis of melanin. While studies have demonstrated the efficacy of Kojic acid for lightening skin, relatively high concentrations (e.g., greater than or equal to 2.0 wt %) are needed. In personal care compositions including relatively lower concentrations (e.g., lower than 2.0 wt %), the Kojic acid has shown only poor to no efficacy in the reduction of melanin and/or the inhibition of melanin production. The poor or no efficacy of Kojic acid at lower concentrations has often been attributed to its chemical and light instability, which leads to reduced delivery of topically applied Kojic acid.

What is needed, then, are improved whitening solid cleansing compositions and delivery vehicles thereof, and methods for the same.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The foregoing and/or other aspects and utilities embodied in the present disclosure may be achieved by providing a solid cleansing composition including a delivery vehicle and one or more whitening agents. The whitening agents may include one or more of kojic acid, phenylethyl resorcinol, or combinations thereof. The whitening agents may be configured to regulate melanin in or on skin.

In at least one implementation, the kojic acid may include one or more of 5-Hydroxy-2-(hydroxymethyl)-4H-pyran-4-one, a kojic acid derivative, or combinations thereof.

In at least one implementation, the kojic acid may include 5-Hydroxy-2-(hydroxymethyl)-4H-pyran-4-one or 5-hydroxy-2-hydroxymethl-γ-pyrone.

In at least one implementation, the kojic acid may include encapsulated kojic acid.

In at least one implementation, the kojic acid may be present in an amount of from about 0.005 wt % to about 2 wt %, based on the total weight of the solid cleansing composition.

In at least one implementation, the resorcinol may include one or more of benzene-1,3-diol, a derivative thereof, or combinations thereof.

In at least one implementation, the resorcinol may include phenylethyl resorcinol.

In at least one implementation, the resorcinol may be present in an amount of from about 0.005 wt % to about 2 wt %, based on the total weight of the solid cleansing composition.

In at least one implementation, the whitening agents may include a combination of the kojic acid and the resorcinol.

In at least one implementation, the kojic acid and resorcinol may be present in a weight ratio of from about 0.1:1 to about 10:1.

In at least one implementation, the delivery vehicle may include at least one cleansing component. The cleansing component may include one or more soaps, one or more synthetic surfactants, or combinations thereof.

The foregoing and/or other aspects and utilities embodied in the present disclosure may be achieved by providing a method for preparing any one of the solid cleansing compositions disclosed herein. The method may include contacting the delivery vehicle and the one or more whitening agents with one another.

The foregoing and/or other aspects and utilities embodied in the present disclosure may be achieved by providing a method for treating, decreasing, reducing, or preventing hyperpigmentation of skin. The method may include contacting an effective amount of any one of the solid cleansing compositions disclosed herein with the skin.

The foregoing and/or other aspects and utilities embodied in the present disclosure may be achieved by providing a method for regulating melanin in or on skin. The method may include contacting an effective amount of any one of the solid cleansing compositions disclosed herein with the skin.

The foregoing and/or other aspects and utilities embodied in the present disclosure may be achieved by providing a method for stabilizing the one or more whitening agents of any one of the solid cleansing compositions disclosed herein. The method may include contacting the delivery vehicle and the one or more whitening agents with one another. Stabilizing the one or more whitening agents may include preventing or inhibiting degradation of any one or more of the whitening agents.

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### DETAILED DESCRIPTION

The following description of various typical aspect(s) is merely exemplary in nature and is in no way intended to limit the disclosure, its application, or uses.

As used throughout this disclosure, ranges are used as shorthand for describing each and every value that is within the range. It should be appreciated and understood that the description in a range format is merely for convenience and brevity, and should not be construed as an inflexible limitation on the scope of any embodiments or implementations disclosed herein. Accordingly, the disclosed range should be construed to have specifically disclosed all the possible subranges as well as individual numerical values within that range. As such, any value within the range may be selected as the terminus of the range. For example, description of a range such as from 1 to 5 should be considered to have specifically disclosed subranges such as from 1.5 to 3, from 1 to 4.5, from 2 to 5, from 3.1 to 5, etc., as well as individual numbers within that range, for example, 1, 2, 3, 3.2, 4, 5, etc. This applies regardless of the breadth of the range.

Unless otherwise specified, all percentages and amounts expressed herein and elsewhere in the specification should be understood to refer to percentages by weight. The amounts given are based on the active weight of the material.

Additionally, all numerical values are “about” or “approximately” the indicated value, and take into account experimental error and variations that would be expected by a person having ordinary skill in the art. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

As used herein, “free” or “substantially free” of a material may refer to a composition, component, or phase where the material is present in an amount of less than 10.0 weight %, less than 5.0 weight %, less than 3.0 weight %, less than 1.0 weight %, less than 0.1 weight %, less than 0.05 weight %, less than 0.01 weight %, less than 0.005 weight %, or less than 0.0001 weight % based on a total weight of the composition, component, or phase.

All references cited herein are hereby incorporated by reference in their entireties. In the event of a conflict in a definition in the present disclosure and that of a cited reference, the present disclosure controls.

The present inventors have surprisingly and unexpectedly discovered that solid cleansing compositions (e.g., bar soap composition) including phenylethyl resorcinol, significantly increased melanin production in the epidermis. The present inventors have also surprisingly and unexpectedly discovered that solid cleansing compositions (e.g., bar soap composition) including a combination of phenylethyl resorcinol and kojic acid significantly reduced melanin in the epidermis. Particularly, the present inventors have surprisingly and unexpectedly discovered that solid cleansing compositions including about 0.01 wt % phenylethyl resorcinol and about 0.05 wt % kojic acid significantly reduced melanin in the epidermis. The present inventors have surprisingly and unexpectedly discovered that solid cleansing compositions including kojic acid and phenylethyl resorcinol in a weight ratio of about 5:1 significantly reduced melanin in the epidermis.

### Compositions

Compositions disclosed herein may be or include solid cleansing compositions. Illustrative solid cleansing compositions may be or include, but are not limited to, soap bars, cleansing bars, and other solid cleansing compositions that may be used for personal cleansing. As used herein, the term “soap bar” or “bar soap” may refer to solid or semi-solid articles for washing, bathing, and cleaning that include soap surfactants, synthetic surfactants, or mixtures thereof. Illustrative soap bars may be or include, but are not limited to, laundry bars, syndet bars, detergent bars, combo bars (combars), fatty acid soap bars, tallow base soap bars, veggie soap bars, or the like.

The solid cleansing compositions may include a delivery vehicle and one or more whitening agents (e.g., skin lightening agents). The delivery vehicle may be or include a cleansing component (e.g., soaps and/or synthetic surfactants) capable of or configured to contain the one or more whitening agents. The delivery vehicle or the cleansing component thereof may also be capable of or configured to deliver (e.g., deposit) the one or more whitening agents on or to skin. For example, the delivery vehicle or the cleansing component thereof may be capable of or configured to provide enhanced or improved delivery of the one or more whitening agents on or to skin as compared to conventional solid cleansing compositions. As further discussed herein, each of the one or more whitening agents may be present in the solid cleansing composition in an effective amount sufficient to regulate or modify (e.g., increase or decrease) melanin in and/or on skin. For example, each of the one or more whitening agents may be present in an effective amount to reduce melanin production in and/or on skin, prevent production of melanin in skin, inhibit the production of melanin in skin, reduce the amount of melanin in and/or on skin, or a combination thereof.

The compositions disclosed herein may have a natural origin index of greater than about 80%. As used herein, the term or expression “natural origin index” may refer to a value (e.g., a percentage) or degree of a substance (e.g., a product or a composition thereof) that describes the natural content of ingredients or formulation of the substance. The natural origin index may be determined by the ISO 16128-2:2017 standard of the International Organization for Standardization (ISO). In at least one example, the natural origin index of the personal care compositions disclosed herein may be greater than 80%, greater than 85%, greater than 90%, greater than 95%, greater than 96%, greater than 97%, greater than 98%, greater than 99%, greater than 99.5%, or 100%.

In at least one implementation, the one or more whitening or skin lightening agents may be or include kojic acid. As used herein, kojic acid may refer to kojic acid, any one or more derivatives thereof, or combinations thereof. Kojic acid is a compound (i.e., 5-Hydroxy-2-(hydroxymethyl)-4H-pyran-4-one or 5-hydroxy-2-hydroxymethl-γ-pyrone) that may be produced by several species of fungi, such as those of the Aspergillus genus. Kojic acid exhibits relatively low stability as it is highly reactive; and thus, degrades over time (e.g., within weeks). The degradation process is often accelerated by heat, UV radiation, low pH, or combinations thereof. In view of the foregoing, relatively high concentrations of kojic acid are needed in conventional solid cleansing compositions to sufficiently function as a tyrosinase inhibitor. For example, kojic acid in concentrations greater than or equal to about 2.0 wt % are needed in conventional solid cleansing compositions to sufficiently function as a tyrosinase inhibitor.

The derivatives of kojic acid may be or include, but are not limited to, kojic acid salts, kojic acid esters, kojic acid polymers, encapsulated kojic acid, or the like, or any combination thereof. Illustrative derivatives of kojic acid may be or include, but are not limited to, kojic acid incorporated into a polymer, such as a degradable polymer (e.g., a poly(carbonate-ester), kojic acid incorporated into a polymer backbone, kojic acid mono-γ-linolenate[2-(6′,9′,12′-octadecatrienoyl)oxymethyl-5-hydroxy-4H-pyran-4-one], isokojic acid, monoesters of kojic acid with fatty acids, diesters of kojic acid with fatty acids, phosphorus derivatives of kojic acid, any one or more kojic acid salts, or the like, or combinations thereof.

The kojic acid may be present in the solid cleansing composition in an amount sufficient to reduce melanin production in and/or on skin, prevent production of melanin in skin, inhibit the production of melanin in skin, reduce the amount of melanin in and/or on skin, or a combination thereof. For example, the kojic acid may be present in the solid cleansing composition in an amount sufficient to reduce melanin production in and/or on skin, prevent production of melanin in skin, inhibit the production of melanin in skin, reduce the amount of melanin in and/or on skin, or a combination thereof. In at least one implementation, the kojic acid is present in an amount sufficient to interact synergistically with another one or more of the whitening agents to reduce melanin production in and/or on skin, prevent production of melanin in skin, inhibit the production of melanin in skin, reduce the amount of melanin in and/or on skin, or a combination thereof.

In an exemplary implementation, the kojic acid may be present in an amount of from about 0.005 wt % to about 2 wt %, based on the total weight of the solid cleansing composition. For example, the kojic acid may be present in an amount of from about 0.005 wt %, about 0.006 wt %, about 0.007 wt %, about 0.008 wt %, about 0.009 wt %, about 0.01 wt %, about 0.015 wt %, about 0.02 wt % about 0.03 wt %, about 0.04 wt %, about 0.05 wt %, about 0.06 wt %, about 0.07 wt %, about 0.08 wt %, about 0.09 wt %, about 0.1 wt %, or more to about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, or about 2.0 wt %, based on the total weight of the solid cleansing composition. In another example, the kojic acid may be present in an amount of from greater than or equal to about 0.005 wt %, greater than or equal to about 0.006 wt %, greater than or equal to about 0.007 wt %, greater than or equal to about 0.008 wt %, greater than or equal to about 0.009 wt %, greater than or equal to about 0.01 wt %, greater than or equal to about 0.015 wt %, greater than or equal to about 0.02 wt % greater than or equal to about 0.03 wt %, greater than or equal to about 0.04 wt %, greater than or equal to about 0.05 wt %, greater than or equal to about 0.06 wt %, greater than or equal to about 0.07 wt %, greater than or equal to about 0.08 wt %, greater than or equal to about 0.09 wt %, greater than or equal to about 0.1 wt %, greater than or equal to about 0.2 wt %, greater than or equal to about 0.3 wt %, greater than or equal to about 0.4 wt %, greater than or equal to about 0.5 wt %, greater than or equal to about 0.6 wt %, greater than or equal to about 0.7 wt %, greater than or equal to about 0.8 wt %, greater than or equal to about 0.9 wt %, greater than or equal to about 1 wt %, greater than or equal to about 1.1 wt %, greater than or equal to about 1.2 wt %, greater than or equal to about 1.3 wt %, or greater than or equal to about 1.4 wt % to about 2.0 wt %, based on the total weight of the solid cleansing composition.

In at least one implementation, the one or more whitening or skin lightening agents may be or include resorcinol. As used herein, the term or expression “resorcinol” may refer to resorcinol (C₆H₄(OH)₂; Benzene-1,3-diol), a derivative thereof, or any combination thereof. Illustrative derivatives of resorcinol may be or include, but are not limited to, butyl resorcinol, pentyl resorcinol, hexyl resorcinol, heptyl resorcinol, octyl resorcinol, nonyl resorcinol, decylresorcinol, phenylethyl resorcinol, phenylpropyl resorcinol, phenylbutyl resorcinol, phenylhexyl resorcinol, toluoylethyl resorcinol, toluoylpropyl resorcinol, toluoylbutyl resorcinol, toluoylhexyl resorcinol, methoxytoluoylethyl resorcinol, methoxytoluoylpropyl resorcinol, methoxytoluoylbutyl resorcinol, methoxyltoluoylhexyl resorcinol, dimethoxytoluoylethyl resorcinol, dimethoxytoluoylpropyl resorcinol, dimethoxytoluoylbutyl resorcinol, dimethoxytoluoylhexyl resorcinol, or the like, or any combination thereof. In an exemplary implementation, the resorcinol derivative may be or include at least phenylethyl resorcinol.

The resorcinol (e.g., resorcinol, a derivative thereof, or any combination thereof) may be present in the solid cleansing composition in an amount sufficient to reduce melanin production in and/or on skin, prevent production of melanin in skin, inhibit the production of melanin in skin, reduce the amount of melanin in and/or on skin, or a combination thereof. For example, the resorcinol may be present in the solid cleansing composition in an amount sufficient to reduce melanin production in and/or on skin, prevent production of melanin in skin, inhibit the production of melanin in skin, reduce the amount of melanin in and/or on skin, or a combination thereof. In at least one implementation, the resorcinol is present in an amount sufficient to interact synergistically with another one or more of the whitening agents to reduce melanin production in and/or on skin, prevent production of melanin in skin, inhibit the production of melanin in skin, reduce the amount of melanin in and/or on skin, or a combination thereof.

In an exemplary implementation, the resorcinol, a derivative thereof, or any combination thereof may be present in an amount of from about 0.005 wt % to about 2 wt %, based on the total weight of the solid cleansing composition. For example, the resorcinol, a derivative thereof, or any combination thereof may be present in an amount of from about 0.005 wt %, about 0.006 wt %, about 0.007 wt %, about 0.008 wt %, about 0.009 wt %, about 0.01 wt %, about 0.015 wt %, about 0.02 wt % about 0.03 wt %, about 0.04 wt %, about 0.05 wt %, about 0.06 wt %, about 0.07 wt %, about 0.08 wt %, about 0.09 wt %, about 0.1 wt %, or more to about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, or about 2.0 wt %, based on the total weight of the solid cleansing composition. In another example, the resorcinol, a derivative thereof, or any combination thereof may be present in an amount of from greater than or equal to about 0.005 wt %, greater than or equal to about 0.006 wt %, greater than or equal to about 0.007 wt %, greater than or equal to about 0.008 wt %, greater than or equal to about 0.009 wt %, greater than or equal to about 0.01 wt %, greater than or equal to about 0.015 wt %, greater than or equal to about 0.02 wt % greater than or equal to about 0.03 wt %, greater than or equal to about 0.04 wt %, greater than or equal to about 0.05 wt %, greater than or equal to about 0.06 wt %, greater than or equal to about 0.07 wt %, greater than or equal to about 0.08 wt %, greater than or equal to about 0.09 wt %, greater than or equal to about 0.1 wt %, greater than or equal to about 0.2 wt %, greater than or equal to about 0.3 wt %, greater than or equal to about 0.4 wt %, greater than or equal to about 0.5 wt %, greater than or equal to about 0.6 wt %, greater than or equal to about 0.7 wt %, greater than or equal to about 0.8 wt %, greater than or equal to about 0.9 wt %, greater than or equal to about 1 wt %, greater than or equal to about 1.1 wt %, greater than or equal to about 1.2 wt %, greater than or equal to about 1.3 wt %, or greater than or equal to about 1.4 wt % to about 2.0 wt %, based on the total weight of the solid cleansing composition.

In an exemplary implementation, the one or more whitening or skin lightening agents include a combination of kojic acid (i.e., kojic acid, a derivative thereof, or combinations thereof) and resorcinol (i.e., resorcinol, a derivative thereof, or combinations thereof). The resorcinol may be or include phenylethyl resorcinol. The kojic acid and the resorcinol may be present in an amount sufficient to interact synergistically with one another to reduce melanin production in and/or on skin, prevent production of melanin in skin, inhibit production of melanin in skin, reduce the amount of melanin in and/or on skin, or combinations thereof. In an exemplarily implementation, the kojic acid and the resorcinol may be present in a weight ratio of from about 0.1:1 to about 10:1. For example, the kojic acid and the resorcinol may be present in a weight ratio of from about 0.1:1, about 0.5:1, about 1:1, about 1.5:1, about 2:1, about 2.5:1, about 3:1, about 3.5:1, about 4:1, about 4.5:1, or about 5:1 to about 5.5:1, about 6:1, about 6.5:1, about 7:1, about 7.5:1, about 8:1, about 8.5:1, about 9:1, about 9.5:1, or about 10:1. In another example, the kojic acid and the resorcinol may be present in a weight ratio of about 0.1:1 to about 10:1, about 2:1 to about 8:1, about 4:1 to about 6:1, or about 5:1.

The solid cleansing composition may include the delivery vehicle. The delivery vehicle may be or include at least one cleansing component (e.g., soaps and/or synthetic surfactants). For example, the solid cleansing composition or the delivery vehicle thereof may include at least one cleansing component. The delivery vehicle or the cleansing component thereof may be capable of or configured to contain the one or more whitening agents and/or effectively or sufficiently deliver the one or more whitening agents to or on skin. In at least one implementation, the delivery vehicle or the cleansing component thereof may be capable of or configured to stabilize the one or more whitening agents. For example, the delivery vehicle or the cleansing component thereof may be capable of or configured to prevent or inhibit chemical and/or light degradation of the one or more whitening agents. For example, the delivery vehicle or the cleansing component thereof may be capable of or configured to prevent or inhibit chemical and/or light degradation of at least one of kojic acid, resorcinol, or combinations thereof. In another implementation, the delivery vehicle or the cleansing component thereof may be capable of or configured to enhance delivery of the one or more whitening agents to skin. For example, the delivery vehicle or the cleansing component thereof may be capable of or configured to deliver the one or more whitening agents to skin more effectively than conventional solid cleansing compositions. In another example, the delivery vehicle or the cleansing component thereof may be capable of or configured to prevent or inhibit degradation of the one or more whitening agents, thereby increasing delivery of the whitening agents to skin.

The delivery vehicle or the cleansing component thereof may include one or more soaps (e.g., soap surfactants), one or more synthetic surfactants, or combinations thereof. In at least one implementation, the cleansing component of the solid cleansing composition may include one or more synthetic surfactants in combination with or in place of the soap surfactants. Solid cleansing compositions including only the one or more synthetic surfactants may be referred to as syndet bars or may be referred to as solid cleansing compositions having a syndet base. Solid cleansing compositions including the one or more synthetic surfactants and the soap surfactants may be referred to as combo bars (combars) or may be referred to as solid cleansing compositions having a combination soap base.

The soap or soap surfactants may be or include alkali metal or alkanol ammonium salts of aliphatic alkane- or alkene-monocarboxylic acids, including about 6 to about 22 carbon atoms, about 6 to about 18 carbon atoms, about 12 to about 18 carbon atoms, or about 12 to about 22 carbon atoms. Illustrative soaps that may be utilized in the solid cleansing composition may be or include, but are not limited to, sodium soaps, ammonium soaps, potassium soaps, magnesium soaps, calcium soaps, mono-, di-, and tri-ethanol ammonium cation soaps, or the like, or mixtures thereof. In a preferred implementation, the cleansing component or soap includes a sodium soap; however, it should be appreciated that at least a portion of the soap may also include one or more ammonium soaps, potassium soaps, magnesium soaps, calcium soaps, or the like, or mixtures and combinations thereof. In a preferred implementation, the cleansing component or the soap may be or include, but is not limited to, alkali metal salts of aliphatic (alkanoic or alkenoic) acids having about 8 to about 22 carbon atoms or about 10 to about 20 carbon atoms.

The cleansing component or the soap thereof may be a fatty acid soap. The fatty acid soap may include one or more neutralized fatty acids. Illustrative fatty acids used for the fatty acid soap may be or include, but are not limited to, myristic acid, lauric acid, palmitic acid, oleic acid, stearic acids, or the like, or combinations thereof. Sources of fatty acids may include coconut oil, palm oil, grape seed oil, palm kernel oil, tallow, avocado, canola, corn, cottonseed, olive, hi-oleic sunflower, mid-oleic sunflower, sunflower, palm stearin, palm kernel olein, safflower, babassu oils, or combinations thereof.

The fatty acids may be neutralized with any base to form the soap or fatty acid soap. Illustrative bases may be or include, but are not limited to, sodium hydroxide, potassium hydroxide, triethanolamine, or the like, or mixtures and combinations thereof. In certain implementations, the fatty acid soap may be formed from fatty acids neutralized by two or more bases. In certain embodiments, the bases are sodium hydroxide and triethanolamine. In certain implementations, the molar ratio of sodium hydroxide and triethanolamine is 1:1. In certain implementations, the fatty acids may be or include any one or more of oleic acid, palmitic acid, stearic acid, lauric acid, or combinations thereof. For example, the fatty acid soap may be or include sodium palmitate, sodium oleate, sodium laurate, sodium stearate, or any combination or mixture thereof. In at least one implementation, the fatty acid soap may further include glycerin.

In at least one implementation, the cleansing component or the soap thereof may include soaps having the fatty acid distribution of tallow and/or one or more vegetable oils (e.g., “fatty acid soaps”). The vegetable oil may be or include, but is not limited to, palm oil, palm kernel oil, palm kernel olein, palm stearin oil, palm kernel olein oil, coconut oil, avocado oil, canola oil, corn oil, cottonseed oil, olive oil, high-oleic sunflower oil, mid-oleic sunflower oil, sunflower oil, safflower oil, babassu oil, sweet almond oil, castor oil, canola oil, soybean oil, olive oil, acai oil, andiroba oil, apricot kernel oil, argan oil, passion fruit oil, marula oil, mango oil, shea oil, macadamia nut oil, brazil nut oil, borage oil, copaiba oil, grape seed oil, buriti oil, sesame oil, flaxseed oil or linseed oil, blueberry oil, cranberry oil, blackberry oil, plum oil, raspberry oil, camelina oil, camellia oil, walnut oil, wheat germ oil, calendula oil, cherry kernel oil, cucumber seed oil, papaya oil, aloe vera oil, hemp oil, or the like, or mixtures or combinations thereof.

In at least one implementation, the cleansing component or the soap thereof may include a mixture or blend of about 60 wt % to about 80 wt % C₁₆-C₁₈ fatty acids and about 20 wt % to about 40 wt % C₁₂-C₁₄ fatty acids, based on the cleansing component or the soap thereof. The C₁₆-C₁₈ fatty acids may be obtained from tallow and the C₁₂-C₁₄ fatty acids may be obtained from one or more vegetable oils, such as lauric oils, palm kernel oils, coconut oils, or combinations thereof. In some example, the cleansing component or the soap thereof may be or include a mixture or blend of about 60 wt % to about 80 wt %, or about 65% to about 75%, or about 70% C₁₆-C₁₈ fatty acids, and about 20 wt % to about 40 wt %, about 25% to about 35%, or about 30% C₁₂-C₁₄ fatty acids, based on the total weight of the cleansing component or the soap thereof.

The cleansing component or the soap thereof may be produced by conventional methods where natural fats and oils such as tallow and/or vegetable oils or their equivalents are saponified with an alkali metal hydroxide using procedures known to those skilled in the art. Alternatively, the cleansing component or the soap thereof may be produced by neutralizing one or more fatty acids, such as lauric, myristic, palmitic, or stearic acids with alkali metal hydroxide or carbonate.

The amount or concentration of any one or more of the soaps (e.g., soap surfactant, fatty acid soap) in the cleansing component may vary widely. In at least one implementation, the amount of any one or more of the soaps in the cleansing component may be greater than or equal to 40 weight % and less than or equal to 95 weight %. For example, the amount of any one or more of the soaps in the cleansing component may be from about 40 weight %, about 50 weight %, about 55 weight %, about 60 weight %, about 65 weight %, or about 70 weight % to about 75 weight %, about 80 weight %, about 85 weight %, about 90 weight %, or about 95 weight %. In another implementation, the amount of any one or more of the soaps in the cleansing component is greater than 70 weight % and less than 80 weight %. For example, the amount of any one or more of the soaps in the cleansing component may be from about 70 weight %, about 71 weight %, about 72 weight %, about 73 weight %, about 74 weight %, or about 75 weight % to about 76 weight %, about 77 weight %, about 78 weight %, about 79 weight %, or about 80 weight %.

Illustrative synthetic surfactants as utilized in syndet bases and combo bars may be or include, but are not limited to, anionic surfactants, amphoteric surfactants, nonionic surfactants, zwitterionic surfactants, and cationic surfactants. In at least one implementation, any one or more of the synthetic surfactants may be present in the solid cleansing composition or the cleansing component thereof in an amount of from greater than or equal to about 50 wt % to about 99 wt %, preferably from about 60 wt % to about 95 wt %, and more preferably from about 70 wt % to about 90 wt %, based on the total weight of the solid cleansing composition of the cleansing component thereof. In another implementation, any one or more of the synthetic surfactants may be present in the solid cleansing composition or the cleansing component thereof in an amount of from about 8.0 wt % to about 65.0 wt %, preferably from about 10.0 wt % to about 60.0 wt %, and more preferably from about 10.0 wt % to about 50.0 wt %, based on the total weight of the solid cleansing composition of the cleansing component thereof.

Illustrative anionic surfactants may be or include, but are not limited to, alkyl sulfates, anionic acyl sarcosinates, methyl acyl taurates, N-acyl glutamates, acyl isethionates, alkyl ether sulfates, alkyl sulfosuccinates, alkyl phosphate esters, ethoxylated alkyl phosphate esters, trideceth sulfates, protein condensates, mixtures of ethoxylated alkyl sulfates, or the like, or combinations thereof. Alkyl chains for these surfactants are C₈-C₂₂, preferably C₁₀-C₁₈ and, more preferably, C₁₂-C₁₄ alkyls.

The zwitterionic surfactants may include those which can be broadly described as derivatives of aliphatic quaternary ammonium, phosphonium, and sulfonium compounds, in which the aliphatic radicals may be straight chain or branched, and where one of the aliphatic substituents may contain from about 8 to about 18 carbon atoms and one contains an anionic water-solubilizing group, for example, carboxy, sulfonate, sulfate, phosphate, or phosphonate. Illustrative zwitterionic surfactants may be or include, but are not limited to, 4-[N,N-di(2-hydroxyethyl)-N-octadecylammonio]-butane-1-carboxylate; 5-[S-3-hydroxypropyl-S-hexadecylsulfonio]-3 hydroxypentane-1-sulfate; 3-[P,P—P-diethyl-P 3,6,9 trioxatetradecyl-phosphonio]-2-hydroxypropane-1-phosphate; 3-[N,N-dipropyl-N-3-dodecoxy-2-hydroxypropylammonio]-propane-1-phosphonate; 3-[N,N-di-methyl-N-hexadecylammonio)propane-1-sulfonate; 3-(N,N-dimethyl-N-hexadecylammonio)-2-hydroxypropane-1-sulfonate; 4-(N,N-di(2-hydroxyethyl)-N-(2 hydroxydodecyl)ammonio]-butane-1-carboxylate; 3-[S-ethyl-S-(3-dodecoxy-2-hydroxypropyl)sulfonio]-propane-1-phosphate; 3-(P,P-dimethyl-P-dodecylphosphonio)-propane-1-phosphonate; 5-[N,N-di(3-hydroxypropyl)-N-hexadecylammonio]-2-hydroxy-pentane-1-sulfate; or the like, or combinations thereof.

The amphoteric surfactants may include those which can be broadly described as derivatives of aliphatic secondary and tertiary amines in which the aliphatic radical may be straight chain or branched and where one of the aliphatic substituents may contain from about 8 to about 18 carbon atoms and one contains an anionic water solubilizing group, e.g., carboxy, sulfonate, sulfate, phosphate, or phosphonate. Illustrative amphoteric surfactant may be or include, but are not limited to, sodium 3-dodecylaminopropionate, sodium 3-dodecylaminopropane sulfonate; N-alkyltaurines, such as the one prepared by reacting dodecylamine with sodium isethionate according to U.S. Pat. No. 2,658,072; N-higher alkyl aspartic acids, such as those produced according to U.S. Pat. No. 2,438,091; and the products sold under the trade name “MIRANOL” and described in U.S. Pat. No. 2,528,378, or the like, or combinations thereof. Other illustrative amphoteric surfactants may be or include, but are not limited to, betaines. The betaines, may be or include, but are not limited to, the high alkyl betaines, such as coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl)carboxy methyl betaine, stearyl bis-(2-hydroxypropyl)carboxymethyl betaine, oleyl dimethyl gamma-carboxypropyl betaine, lauryl bis-(2-hydro-xypropyl)alpha-carboxyet-hyl betaine, etc. The sulfobetaines may be represented by coco dimethyl sulfopropyl betaine, stearyl dimethyl sulfopropyl betaine, amido betaines, amidosulfobetaines, or the like, or combinations thereof.

Illustrative cationic surfactants may be or include, but are not limited to, stearyldimenthylbenzyl ammonium chloride; dodecyltrimethylammonium chloride; nonylbenzylethyldimethyl ammonium nitrate; tetradecylpyridinium bromide; laurylpyridinium chloride; cetylpyridinium chloride; laurylpyridinium chloride; laurylisoquinolium bromide; ditallow(Hydrogenated)dimethyl ammonium chloride; dilauryldimethyl ammonium chloride; stearalkonium chloride; or other cationic surfactants known in the art, or combinations thereof.

The nonionic surfactants may include those broadly defined as compounds produced by the condensation of alkylene oxide groups (hydrophilic in nature) with an organic hydrophobic compound, which may be aliphatic or alkyl aromatic in nature.

In a preferred implementation, the synthetic surfactants of the cleansing component may include sodium cocoyl isethionate, disodium lauryl sulfosuccinate, or combinations thereof.

The solid cleansing composition and the cleansing component thereof may include water. Water of the solid cleansing composition and the cleansing component thereof may be deionized water, demineralized water, and/or softened water. Water of the cleansing component may be separate from the water of other components of the solid cleansing composition. For example, water of the soap and/or the synthetic surfactants may be separate from water in other components of the solid cleansing composition. Water may make up the balance of the solid cleansing composition. For example, the amount of water in the solid cleansing composition may be from about 1 weight % to about 10 weight %, about 10 weight % to about 20 weight %, about 12 weight % to about 18 weight %, or about 14 weight % to about 16 weight %. In another example, the amount of water in the solid cleansing composition may be at least 10 weight %, at least 11 weight %, at least 12 weight %, at least 13 weight %, at least 14 weight %, at least 15 weight %, at least 16 weight %, or at least 17 weight %. In at least one implementation, the amount of water may be about 10 weight %, about 11 weight %, about 12 weight %, about 13 weight %, about 14 weight %, or about 15 weight %. The amount of water in the solid cleansing composition may include free water added and/or water introduced with other components or materials of the solid cleansing composition. For example, the amount of water in the solid cleansing composition may include free water and water associated with the soap, the synthetic surfactant, and/or any other component of the solid cleansing composition.

The solid cleansing composition may include one or more humectants. Illustrative humectants may include, but are not limited to, one or more of ascorbic acid, ascorbyl dipalmitate, acetamide MEA or acetamide monoethanolamine, glucose glutamate, glucuronic acid, triethanolamine salt of lactic acid (TEA-lactate), pyroglutamic acid triethanolamine salt (TEA-PCA), corn syrup, fructose, glucose, glycerin, glycol, 1,2,6-hexanetriol, sodium lactate, sodium salt of pyroglutamic acid (sodium PCA), hydrogenated starch hydrolysate, inositol, lactic acid, lactose, mannitol, pyroglutamic acid (PCA), PEG-10 propylene glycol, polyamino sugar condensate, propylene glycol, pyridoxine dilaurate, saccharide hydrolysate, hydroxystearyl methylglucamine, glucamine, maltitol, mannitol, methyl gluceth-10, methyl gluceth-20, riboflavin, PEG-4, PEG-6, PEG-8, PEG-9, PEG-10, PEG-12, PEG-14, PEG-16, PEG-18, PEG-20, PEG-32, PEG-40, where PEG are polymers of ethylene glycol, glutamic acid, glycereth-7, glycereth-12, glycereth-26, saccharide isomerate, sorbeth-20, sorbitol, sucrose, thioglycerin, tris-(hydroxymethyl)nitromethane, tromethamine, histidine, PEG-75, PEG-135, PEG-150, PEG-200, PEG-5 pentaerythritol ether, polyglyceryl sorbitol, sorbitol, urea, xylitol, or the like, or combinations thereof. In a preferred implementation, the solid cleansing composition includes glycerin.

In at least one implementation, the solid cleansing composition may include one or more free fatty acids configured to provide enhanced skin feel benefits. For example, the solid cleansing composition may include the fatty acids to provide softer or smoother feeling skin. Illustrative fatty acids may include, but are not limited to, fatty acids of palm kernel oil, palm oil, coconut oil, olive oil, laurel oil, or the like, or combinations thereof. Illustrative fatty acids may also include animal fats, such as tallow. Illustrative fatty acids may also include, but are not limited to, fatty acid sources having fatty acid distributions similar or substantially similar to natural or synthetic fatty acid sources (e.g., natural animal fats or oils, natural vegetable fats or oils, individual fatty acids, etc.). The free fatty acids may be separate from the fatty acids associated with the plant oils. In a preferred implementation, the fatty acid sources may be natural fatty acid sources.

The free fatty acids may be present in the solid cleansing composition in an amount of from about 2 wt % to about 15 wt %, based on the total weight of the solid cleansing composition. For example, the free fatty acids may be present in an amount of from about 2 wt %, about 3 wt %, about 4 wt %, about 5 wt %, about 6 wt %, or about 7 wt % to about 8 wt %, about 9 wt %, about 10 wt %, about 11 wt %, about 12 wt %, about 13 wt %, about 14 wt %, or about 15 wt %, based on the total weight of the solid cleansing composition. In another example, the free fatty acids may be present in an amount of from about 2 wt % to about 15 wt %, about 5 wt % to about 10 wt %, preferably about 6 wt % to about 9 wt %, or more preferably about 7 wt % to about 8 wt %, based on the total weight of the solid cleansing composition.

The solid cleansing composition may include one or more skin care agents. Any suitable skin care agents that do not adversely affect the stability and/or efficacy of the solid cleansing composition may be used. In at least one implementation, the skin care agent may include an emollient configured to maintain a soft, smooth, and pliable appearance to the skin. As is known by those skilled in the art, the emollients may function by remaining on the surface of the skin or in the stratum corneum to act as a lubricant, to reduce flaking, and/or to improve the appearance of the skin.

The skin care agents may generally include one or more polymers (e.g., polyvinylpyrrolidine), protein derivatives (e.g., derivatized hydrolyzed wheat protein), ethoxylated fatty ethers, cellulosics (e.g., hydroxyethylcellulose), or the like, or mixtures and combinations thereof. Illustrative skin care agents may include, but are not limited to, esters comprising an aliphatic alcohol having about 2 to about 18 carbon atoms condensed with an aliphatic or aromatic carboxylic acid including about 8 to about 20 carbon atoms (e.g., isopropyl myristate, decyl oleate, cetearyl isononanate, etc.). The esters may be straight chained or branched. In a preferred implementation, the ester has a molecular weight of less than about 500.

Other skin care agents may include, but are not limited to, polyvinyl-pyrrolidone, polyquaternium-4, polyquaternium-6, polyquaternium-7, polyquaternium-10, guar gum derivatives, hydroxypropylmethylcellulose, hydroxyethylcellulose, a polyethylene glycol, a methyl ether of a polyethylene glycol, quaternium-79, wheat germamidopropyl hydroxypropyl dimonium hydrolyzed wheat protein, stearyl methicone, dimethicone copolyol, dimethicone propyl PG betaine, poly(sodium styrene sulfonate), sorbitan oleate, steareth-2, steareth-21, isoceteth-20, PEG-7 glyceryl cocoate, PEG-75 lanolin, glycereth-26, PPG-5-ceteth-20, a C₁₂-C₂₀ alcohol, canola oil, glyceryl laurate, triglyceryl monostearate, glyceryl monostearate, vitamin E acetate, sunflower seed amidopropylethyldimonium ethylsulfate, sodium PEG-7 olive oil carboxylate, PPG-1 hydroxyethyl caprylamide, PPG-2 hydroxyethyl cocamide, mineral oil, petrolatum, aloe barbadensis, isostearamidopropylmorpholine lactate, strontium acetate, palmitamidopropyltrimonium chloride, or the like, or combinations thereof. In a preferred implementation, the skin care agent excludes, is free, or substantially free of the polyquats (e.g., polyquaternium-4/6/7/10, etc.).

The solid cleansing composition may include one or more salts capable of or configured to modify the one or more surfactants of the solid cleansing composition. For example, the salts may be configured to at least partially modify a cloud point of the surfactants to thereby control the haze or transparency of the cleansing composition. The salts may be or include one or more inorganic salts including, but not limited to, sodium sulfate, magnesium sulfate, sodium chloride, sodium citrate, or the like, or combinations thereof. The amount of any one or more of the salts may be at least partially determined by the type and/or amount of the surfactants included in the solid cleansing composition. In at least one implementation, the amount of any one or more of the salts may be about 0.1 weight %, 0.2 weight %, 0.3 weight %, 0.4 weight %, or 0.5 weight % to about 0.6 weight %, 0.7 weight %, 0.8 weight %, 0.9 weight %, or about 1.0 weight %.

The solid cleansing composition may include one or more additional ingredients. Illustrative ingredients may include, but are not limited to, one or more dyes, fragrances (e.g., limonene, ethyl butyrate, linalool, and/or oils, such as citronellol, coumarin, benzyl salicylate, etc.), buffers and buffering agents (e.g., inorganic phosphates, sulfates, and carbonates), pH adjusters (e.g., acids and/or bases), preservatives (e.g., parabens, hydantoins, imidazolines, etc.), thickeners, viscosity modifiers, antioxidants (e.g., etidronic acid, etc.), foam enhancers, chelating agents (e.g., EDTA, phosphates, pentasodium pentetate, etidronic acid, etc.), skin conditioning agents, opacifiers, hydric solvents, hydrotropes, antimicrobials, sunscreen actives, anti-aging compounds, vitamins, essential oils and extracts (e.g., rosewood, jojoba, etc.), polyols, titanium dioxide, abrasives (e.g., particulate matter), acaricidal agents (e.g., benzyl benzoate), or the like, or combinations thereof.

Illustrative antimicrobials may be or include, but are not limited to, trichlorocarbanilide (TCC), triclosan, geraniol, carvacrol, citral, eucalyptol, catechol, 4-allylcatechol, hexyl resorcinol, methyl salicylate, triclocarban, or the like, or combinations thereof. Illustrative anti-aging compounds may include, but are not limited to, alpha hydroxy acids, beta hydroxy acids, polyhydroxy acids, or the like, or combinations thereof. Illustrative sunscreen actives may include, but are not limited to, butyl methoxy benzoylmethane, or the like, or combinations thereof. Illustrative polyols may include, but are not limited to, glycerol, sorbitol, propylene glycol, polyethylene glycol, or the like, or combinations thereof. Illustrative abrasives or particulate matter may include, but are not limited to, silica, talc, calcium carbonate, polyethylene beads, jojoba beads, lufa, oat flour, or the like, or combinations thereof. Illustrative vitamins may include, but are not limited to, vitamins such as vitamin A, E, K, C, or combinations thereof.

The solid cleansing composition may include preservatives in an amount greater than 0.00 weight % and less than or equal to about 3.0 weight % or less than or equal to about 2.0 weight %. Illustrative preservatives may include, but are not limited to, benzalkonium chloride; benzethonium chloride, 5-bromo-5-nitro-1,3-dioxane; 2-bromo-2-nitropropane-1,3-diol; alkyl trimethyl ammonium bromide; N-(hydroxymethyl)-N-(1,3-dihydroxy methyl-2,5-dioxo-4-imidaxolidinyl-N-(hydroxy methyl)urea; 1-3-dimethyol-5,5-dimethyl hydantoin; formaldehyde; iodopropynl butyl carbamate, butyl paraben; ethyl paraben; methyl paraben; propyl paraben, mixture of methyl isothiazolinone/methyl-chloroisothiazoline in a 1:3 wt. ratio; mixture of phenoxythanol/butyl paraben/methyl paraben/propylparaben; 2-phenoxyethanol; tris-hydroxyethyl-hexahydrotriaz-ine; methylisothiazolinone; 5-chloro-2-methyl-4-isothiazolin-3-one; 1,2-dibromo-2,4-dicyanobutane; 1-(3-chloroalkyl)-3,5,7-triaza-azoniaadam-antane chloride; sodium benzoate; organic acids, sorbic acid, lactic acid, citric acid, or the like, or combinations thereof.

The solid cleansing composition may include one or more plant oils. As used herein, “plant oil” may refer to oil that is obtained from a plant, or manufactured oil made by blending at least two components of oil (e.g., triglycerides, saturated and/or unsaturated fatty acids, etc.) to substantially mimic the composition of a natural plant oil or provide an oil substantially similar in composition to a plant oil. For example, a manufactured oil substantially similar in composition to a plant oil may include at least 50 weight %, at least 60 weight %, at least 70 weight %, at least 80 weight %, at least 90 weight %, at least 95 weight %, at least 98 weight %, at least 99 weight %, at least 99.5 weight %, at least 99.9 weight %, or 100 weight % of the components that are naturally found in the plant oil that the manufactured oil is designed to substantially mimic.

Illustrative plant oils may be or include, but are not limited to, palm oil, palm kernel oil, palm kernel olein, palm stearin oil, palm kernel olein oil, coconut oil, avocado oil, canola oil, corn oil, cottonseed oil, olive oil, high-oleic sunflower oil, mid-oleic sunflower oil, sunflower oil, safflower oil, babassu oil, sweet almond oil, castor oil, canola oil, soybean oil, olive oil, acai oil, andiroba oil, apricot kernel oil, argan oil, passion fruit oil, marula oil, mango oil, shea oil, macadamia nut oil, brazil nut oil, borage oil, copaiba oil, grape seed oil, buriti oil, sesame oil, flaxseed oil or linseed oil, blueberry oil, cranberry oil, blackberry oil, plum oil, raspberry oil, camelina oil, camellia oil, walnut oil, wheat germ oil, calendula oil, cherry kernel oil, cucumber seed oil, papaya oil, aloe vera oil, hemp oil, or the like, or mixtures or combinations thereof. In a preferred implementation, the plant oil includes at least palm oil.

The one or more plant oils may be or include components of any one or more of the plant oils. For example, in at least one implementation, the plant oils may include one or more components or fractions of the plant oil. For example, palm oil may include a liquid fraction, referred to as palm olein, and a solid fraction, referred to as palm stearin. In a preferred implementation, the one or more plant oils may include at least palm oil, palm stearin, palm kernel olein, or combinations thereof.

The amount or concentration of the one or more plant oils present in the solid cleansing composition may vary widely. In at least one implementation, the amount of the one or more plant oils present in the solid cleansing composition may be from greater than 0 weight % to less than or equal to 5 weight %, based on the total weight of the solid cleansing composition. For example, the amount of the one or more plant oils present in the solid cleansing composition may be from greater than 0 weight %, about 0.5 weight %, about 1 weight %, about 1.5 weight %, about 2 weight %, or about 2.5 weight % to about 3 weight %, about 3.5 weight %, about 4 weight %, about 4.5 weight %, or about 5 weight %, based on the total weight of the solid cleansing composition. In another example, the amount of the one or more plant oils present in the solid cleansing composition may be from greater than 0 weight % to about 5 weight %, about 0.5 weight % to about 4.5 weight %, about 1 weight % to about 4 weight %, about 1.5 weight % to about 3.5 weight %, or about 2 weight % to about 3 weight %. In another implementation, the amount of the one or more plant oils present in the solid cleansing composition may be from greater than 0 weight % to less than or equal to 1 weight %, based on a total weight of the solid cleansing composition. For example, the amount of the one or more plant oils present in the solid cleansing composition may be from greater than 0 weight %, about 0.1 weight %, about 0.2 weight %, about 0.3 weight %, about 0.4 weight %, or about 0.45 weight % to about 0.5 weight %, about 0.6 weight %, about 0.7 weight %, about 0.8 weight %, about 0.9 weight %, or about 1.0 weight %. In another example, the amount of the one or more plant oils present in the solid cleansing composition may be from greater than 0 weight % to about 1.0 weight %, about 0.1 weight % to about 0.9 weight %, about 0.2 weight % to about 0.8 weight %, about 0.3 weight % to about 0.7 weight %, about 0.4 weight % to about 0.6 weight %, or about 0.45 weight % to about 0.5 weight %.

The solid cleansing composition may include one or more hydrolyzed proteins. The hydrolyzed proteins may be completely hydrolyzed or partially hydrolyzed. Illustrative hydrolyzed proteins may be or include, but are not limited to, hydrolyzed gelatin, hydrolyzed collagen, hydrolyzed casein, hydrolyzed whey protein, hydrolyzed milk protein, hydrolyzed soy protein, hydrolyzed egg protein, hydrolyzed wheat protein, amino acids, peptides, and the like, or combinations thereof. In a preferred implementation the hydrolyzed protein includes hydrolyzed milk protein, such as CAS 92797-39-2 (EINECS: 296-575-2). For example, the hydrolyzed protein may be or include a hydrolyzed phosphoprotein derived from natural dairy protein, such as MILK TEIN NPNF®, which is commercially available from Tri-K Industries Inc. of Denville, N.J.

The hydrolyzed milk protein may be or include milk protein hydrolyzed by an enzyme. For example, a milk protein may be enzymatically hydrolyzed to provide the hydrolyzed milk protein.

The hydrolyzed protein may be a solution or mixture. In one implementation, the hydrolyzed protein may be provided as a pure or substantially pure solution. In another implementation, the hydrolyzed protein may be provided as a solution including one or more hydrolyzed proteins dissolved, mixed, or otherwise dispersed in the solution. In at least one implementation, the hydrolyzed protein may be an aqueous solution including the hydrolyzed protein in an amount of from greater than 0 weight % to less than or equal to 50 weight %. For example, a hydrolyzed protein solution may be an aqueous solution including one or more hydrolyzed proteins in an amount of from greater than 0 weight %, about 5 weight %, about 10 weight %, about 15 weight %, about 18 weight %, or about 20 weight % to about 25 weight %, about 30 weight %, about 35 weight %, about 40 weight %, about 45 weight %, or about 50 weight %. In another example, the hydrolyzed protein solution may be an aqueous solution including one or more hydrolyzed proteins in an amount of from greater than 0 weight % to about 50 weight %, about 5 weight % to about 45 weight %, about 10 weight % to about 40 weight %, about 15 weight % to about 35 weight %, about 20 weight % to about 30 weight %, or about 20 weight % to about 25 weight %. In a preferred implementation, the hydrolyzed protein solution is an aqueous solution including about 22 weight % of the hydrolyzed protein in water. In a more preferred implementation, the hydrolyzed protein solution is an aqueous solution including about 22 weight % of hydrolyzed milk protein in water.

The amount or concentration of the hydrolyzed protein solution present in the solid cleansing composition may vary widely. In at least one implementation, the amount of the hydrolyzed protein solution (e.g., a 22 weight % solution of hydrolyzed proteins) present in the solid cleansing composition may be from greater than 0 weight % to less than or equal to 10 weight %, based on the total weight of the solid cleansing composition. For example, the amount of the hydrolyzed protein solution present in the solid cleansing composition may be from greater than 0 weight %, about 1 weight %, about 2 weight %, about 3 weight %, about 4 weight %, or about 5 weight % to about 6 weight %, about 7 weight %, about 8 weight %, about 9 weight %, or about 10 weight %, based on a total weight of the solid cleansing composition. In another example, amount of the hydrolyzed protein solution present in the solid cleansing composition may be from greater than 0 weight %, about 0.2 weight %, about 0.4 weight %, about 0.6 weight %, about 0.8 weight % or about 1.0 weight % to about 1.2 weight %, about 1.4 weight %, about 1.6 weight %, about 1.8 weight %, or about 2.0 weight %, based on the total weight of the solid cleansing composition.

The amount or concentration of the one or more hydrolyzed proteins present in the solid cleansing composition may vary widely. In at least one implementation, the amount of the one or more hydrolyzed proteins present in the solid cleansing composition may be from greater than 0 weight % to less than or equal to 1 weight %, based on the total weight of the solid cleansing composition. For example, the amount of the one or more hydrolyzed proteins present in the solid cleansing composition may be from greater than 0 weight %, about 0.05 weight %, about 0.1 weight %, about 0.15 weight %, about 0.2 weight %, about 0.25 weight %, about 0.3 weight %, about 0.35 weight %, about 0.4 weight %, about 0.45 weight %, or about 0.5 weight % to about 0.55 weight %, about 0.6 weight %, about 0.65 weight %, about 0.7 weight %, about 0.75 weight %, about 0.8 weight %, about 0.85 weight %, about 0.9 weight %, about 0.95 weight %, or about 1 weight %, based on the total weight of the solid cleansing composition. In a preferred implementation, the amount of the one or more hydrolyzed proteins present in the solid cleansing composition may be from about 0.15 weight % to about 0.3 weight %, more preferably about 0.2 weight % to about 0.25 weight %, even more preferably about 0.22 weight %, based on the total weight of the solid cleansing composition.

In an exemplary implementation, the solid cleansing composition excludes the one or more hydrolyzed proteins. For example, the solid cleansing composition may be free or substantially free of the hydrolyzed proteins. Accordingly, in an exemplary implementation, the solid cleansing composition may include flaxseed oil and exclude the hydrolyzed proteins.

In an exemplary implementation, the solid cleansing compositions disclosed herein include a delivery vehicle and one or more whitening agents. The delivery vehicle includes soap chips containing about 42.5% palm oil, about 42.5% palm stearin, about 15% palm kernel oil, and minors/excipients. The whitening agents include a combination of phenylethyl resorcinol and kojic acid. The kojic acid and the phenylethyl resorcinol may be present in a weight ratio of about 3:1 to about 7:1, about 4:1 to about 6:1, or about 5:1. The kojic acid may be present in an amount of about 0.03 wt % to about 0.07 wt %, about 0.04 wt % to about 0.06 wt %, or about 0.05 wt %. The phenylethyl resorcinol may be present in an amount of about 0.005 wt % to about 0.015 wt %, about 0.008 wt % to about 0.013 wt %, or about 0.01 wt %.

Unless otherwise specifically identified, the ingredients for use in the compositions and formulations of the compositions disclosed herein are preferably cosmetically acceptable ingredients. As used herein, the expression “cosmetically acceptable” may refer to a component or ingredient that is suitable for use in a formulation for topical application to human skin. A cosmetically acceptable excipient, may refer to an excipient that is suitable for external application in the amounts and concentrations contemplated in the formulations of the compositions disclosed herein, and includes for example, excipients which are “Generally Recognized as Safe” (GRAS) by the United States Food and Drug Administration (FDA).

### Methods

The present disclosure may provide methods for preparing a solid cleansing composition. The method may include mixing, stirring, combining, or otherwise contacting a delivery vehicle (or a cleansing component thereof) and one or more whitening agents with one another. In at least one example, the solid cleansing composition is a bar soap. The one or more whitening agents may be or include kojic acid, phenylethyl resorcinol, or combinations thereof. In at least one implementation, the one or more whitening or lightening agents includes a combination of kojic acid and phenylethyl resorcinol. The delivery vehicle or the cleansing component thereof may include one or more soaps (e.g., fatty acid soaps), one or more synthetic surfactants, or combinations thereof.

The present disclosure may provide methods for treating, decreasing, reducing, or preventing hyperpigmentation of skin, irregular distribution of melanin in or on skin, overproduction of melanin in or on skin, or combinations thereof. The method may include contacting an effective amount of any one or more of the solid cleansing compositions disclosed herein with the skin. For example, the method may include contacting an effective amount of the solid cleansing compositions including a synergistic amount of the delivery vehicle or the cleansing component thereof and/or the one or more whitening agents with the skin. The one or more whitening agents may be or include kojic acid, phenylethyl resorcinol, or combinations thereof. The cleansing component may include one or more soaps (e.g., fatty acid soaps), one or more synthetic surfactants, or combinations thereof.

The present disclosure may provide methods for regulating melanin in and/or on skin. For example, the present disclosure may provide methods for reducing melanin production in and/or on skin, prevent production of melanin in skin, inhibit the production of melanin in skin, reduce the amount of melanin in and/or on skin, or a combination thereof. The method may include contacting an effective amount of any one or more of the solid cleansing compositions disclosed herein with the skin. For example, the method may include contacting an effective amount of the solid cleansing compositions including a synergistic amount of the delivery vehicle or the cleansing component thereof and/or the one or more whitening agents with the skin. The one or more whitening agents may be or include kojic acid, phenylethyl resorcinol, or combinations thereof. The cleansing component may include one or more soaps (e.g., fatty acid soaps), one or more synthetic surfactants, or combinations thereof.

The present disclosure may provide methods for stabilizing the one or more whitening agents in the solid cleansing composition. Stabilizing the one or more whitening agents may include preventing or inhibiting chemical and/or light degradation of the one or more whitening agents. The method may include mixing, stirring, combining, or otherwise contacting the delivery vehicle (or a cleansing component thereof) and the one or more whitening agents with one another.

### EXAMPLES

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

### Example 1

A base bar soap composition or control (1) was prepared by combining soap chips containing 42.5% palm oil, 42.5% palm stearin, 15% palm kernel oil and minors/excipients, according to Table 1. Test bar soap compositions (2) and (3) were then prepared by adding varying amounts of phenylethyl resorcinol and kojic acid, according to Table 2. As indicated in Table 2, the control (1) did not include any amount of either phenylethyl resorcinol or kojic acid. As such, it should be appreciated that any differences between the control bar soap composition (1) and the test bar soap compositions (2) and (3) are attributed to the respective combinations of the phenylethyl resorcinol and kojic acid.

TABLE 1
Composition of Base Bar Soap Composition (1)
Ingredient/Component

Soap chips containing 42.5% palm oil,
42.5% palm stearin, 15% palm kernel oil
Minors/Excipients

TABLE 2
Compositions of Control and Test Bar Soap Compositions (1)-(3)

| | Base Bar Soap | Phenylethyl | |
|---|---|---|---|
| | Composition | Resorcinol | Kojic Acid |
| # | (wt %) | (wt %) | (wt %) |
| (1) Control | 100.0 | 0.0 | 0.0 |
| (2) | 99.94 | 0.01 | 0.05 |
| (3) | 99.92 | 0.03 | 0.05 |

### Example 2

Each of the test bar soap compositions (2) and (3) of Example 1 were evaluated for their efficacy in delivering phenylethyl resorcinol to skin using the base bar soap composition and/or the kojic acid as a vehicle. Porcine back skin with the subcutaneous fat removed was used as skin models for the control bar soap composition (1) and the test bar soap compositions (2) and (3).

To evaluate deposition or delivery of phenylethyl resorcinol to the skin, water at a flowrate of about 6 liters/min and at a temperature of about 38° C. (about 100° F.) was used. Both the skin model sample and each of the bar soap compositions (1)-(3) was wetted or rinsed with water for about five seconds. Then, each of the respective bar soap compositions (1)-(3) was contacted or rubbed onto the skin for about 10 seconds and lathered for an additional 15 seconds. After rubbing and lathering, each of the skin model samples was allowed to rest for about 30 seconds and subsequently rinsed for about 15 seconds. Excess water was then shaken off of the skin model sample and the skin model sample was allowed to air dry for about 5 minutes.

To quantify the amount of phenylethyl resorcinol delivered to each of the skin model samples, a cup scrub method was utilized to extract the phenylethyl resorcinol from the respective skin model samples. Particularly, a glass cup having a diameter of about 3 cm was placed in the center of the skin model sample, about 1 mL of methanol was disposed inside the cup, and a glass rod was used to rub the skin model sample contacting the methanol about 30 seconds. The methanol was then removed with a pipette and disposed in a vial. This process of extraction was repeated or performed in duplicate, thereby resulting in a total of about 2 mL of extracted methanol. Each of the vials containing the extracted methanol were left to dry. After drying, the samples were re-solubilized in about 1.5 mL of a solution including acetonitrile, water, and methanol in a volume ratio of 2:1:2, and subsequently filtered into an HPLC vial.

Each of the samples in the HPLC vial extracted from the respective skin models was then evaluated with a HPLC spectrophotometer with a UV detector. The HPLC spectrophotometer was utilized in the following configuration: a mobile phase of 2/1/2 acetonitrile/water/methanol at a flow rate of about 0.8 mL/min, a reverse phase, Agilent C18 “Zorbax” column (150 mm×4.6 mm) with an attached guard column commercially available from Agilent Technologies, Inc. of Santa Clara, Calif. was utilized, an injection volume of about 20 μL, and a detection wavelength of about 280 nm with a retention time of about 4.75 minutes.

A calibration curve was generated using known serial dilutions. The calibration curve exhibited a linear behavior (R²>0.999). The calculated concentration of phenylethyl resorcinol for each of the samples was then corrected for dilution and total reaction volume. The respective amounts of phenylethyl resorcinol extracted from each of the skin model samples tested with the respective control bar soap composition (1) or the test bar soap compositions (2) and (3) are summarized in Table 3.

TABLE 3
Amount of Phenylethyl Resorcinol Extracted from Skin
Models Treated with Control Bar Soap Composition
(1) and Test Bar Soap Compositions (2) and (3)

| # | Amount of Phenylethyl Resorcinol (μg/cm²) |
|---|---|
| (1) Control | Not detected ^{A} |
| (2) | 0.0034 ± 0.0040 ^{B} |
| (3) | 0.0064 ± 0.0022 ^{B} |

^{A} Sample size of 2 (N = 2)
^{B} Sample size of 4 (N = 4)

It should be appreciated that the relatively high standard deviation for the test bar soap composition (2) was due to a single high value for one of the deposition runs. As indicated in Table 3, almost twice as much phenylethyl resorcinol was detected on skin treated or otherwise contacted with the test bar soap composition (3) including about 0.03 wt % phenylethyl resorcinol as compared to the test bar soap composition (2) including about 0.01 wt % phenylethyl resorcinol. The results clearly demonstrate that the base bar soap composition or control (1) effectively released and delivers phenylethyl resorcinol to skin.

### Example 3

Test bar soap compositions (4)-(7) were prepared by adding varying amounts of phenylethyl resorcinol and kojic acid, according to Table 4. The control bar soap composition (1) of Example 1 and a reference bar soap composition (8) were utilized as comparisons. The reference bar soap composition (8) was prepared by combining soap chips containing 42.5% palm oil, 42.5% palm stearin, 15% palm kernel oil and other components, according to Table 5. As indicated in Table 5, the reference bar soap composition (8) was similar to the control base bar soap composition (1), but also included Extrapone® Papaya commercially available from Symrise of Holzminden, Germany and Campo Songyi Mushroom gel ceramide oil extract as active whitening ingredients. It should be appreciated that both Extrapone® Papaya and Campo Songyi are claimed to have whitening effects on skin.

TABLE 4
Compositions of Control and Test Bar Soap Compositions (1)-(3)

| | Base Bar Soap | Phenylethyl | |
|---|---|---|---|
| | Composition | Resorcinol | Kojic Acid |
| # | (wt %) | (wt %) | (wt %) |
| (1) Control | 100.0 | 0 | 0 |
| (4) | 99.95 | 0 | 0.05^{A} |
| (5) | 99.99 | 0.01 | 0 |
| (6) | 99.94 | 0.01 | 0.05 |
| (7) | 99.98 | 0.01 | 0.01 |

^{A}Encapsulated kojic acid

TABLE 5
Composition of Reference Bar Soap Composition (8)
Ingredient/Component

Soap chips containing 42.5% palm oil,
42.5% palm stearin, 15% palm kernel oil
Extrapone ® Papaya
Campo Songyi Mushroom gel ceramide oil extract

As indicated in Table 4, the control (1) did not include any amount of either phenylethyl resorcinol or kojic acid. As such, it should be appreciated that any differences between the control bar soap composition (1) and the test bar soap compositions (4)-(7) are attributed to the respective combinations of the phenylethyl resorcinol and kojic acid.

### Example 4

Each of the control bar soap composition (1), the test bar soap compositions (4)-(7), and the reference bar soap composition (8) were evaluated for their efficacy in reducing melanin. Specifically, the efficacy of the bar soap compositions (1) and (4)-(8) for reducing melanin in ex vivo human skin models was evaluated. The skin models used to predict human biological response were NativeSkin® Human Skin Models commercially available from GENOSKIN of Salem, Mass.

A total of 21 biopsies having a diameter of about 1 mm were obtained from a single donor. The 21 biopsies were utilized to produce 21 NativeSkin® skin models according to standard procedures. The skin models were cultured under cell culture conditions (about 37° C., 5% CO₂, max. humidity) for 7 days with 1 mL of standard NativeSkin® medium.

Each of the bar soap compositions (1) and (4)-(8) were evaluated by preparing respective solutions. Particularly, each of the bar soap compositions (1) and (4)-(8) was utilized to prepare dilute 5% bar soap solutions with water, which were subsequently diluted to 1% with phosphate-buffered saline (PBS) to prepare test solutions of each of the bar soap compositions (1) and (4)-(8).

To evaluate the efficacy of each of the bar soap compositions (1) and (4)-(8), each of the test solutions were topically applied to respective skin models daily for seven days. Specifically, each daily treatment included contacting about 30 μL of each test solution with respective NativeSkin® skin models, incubating the skin models for about 1 hour at about 37° C. and about 5% CO₂, then subsequently rinsing with 30 μL of PBS eight times. After each rinsing with the PBS, the skin models were dried with a sterile cotton swab. After the final rinsing and drying cycle, the skin models were incubated at about 37° C. and about 5% CO₂ until the next treatment. After treatment for seven days, each of the skin models were collected about four hours after the last rinsing and drying cycle.

Histological analysis was conducted on each of the skin models. Specifically, Fontana-Masson staining was performed on 5 μm thickness paraffin-embedded skin cross-sections. For each sample, a whole scan of the epidermis/dermis was acquired with a 20× lens by iHisto®, Inc. of Salem Mass. Quantification of the positive staining was performed by QuantaCell of Pessac, France. The area of melanin reported to epidermis area±standard error is summarized in Table 6.

TABLE 6
Quantification of Melanin

| | Phenylethyl | Kojic | |
|---|---|---|---|
| | Resorcinol | Acid | Melanin |
| # | (wt %) | (wt %) | (%) |
| Untreated Skin | N/A | N/A | 100 |
| (1) Control | 0 | 0 | 88.44 |
| (4) | 0 | 0.05 | 101.14 |
| (5) | 0.01 | 0 | 110.00 |
| (6) | 0.01 | 0.05 | 68.43 |
| (7) | 0.01 | 0.01 | 81.92 |
| (8) Reference | — | — | 104.83 |

The amount of melanin in each of the skin models treated with the test bar soap compositions (4)-(7) were compared to the skin model treated with the control bar soap composition (1), and p-values were determined for significance (p-value≤0.05). The results are summarized in Table 7.

TABLE 7
Quantification of Melanin in Skin Model Treated with
Test Bar Soap Compositions (4)-(7) Compared with Skin
Model Treated with Control Bar Soap Composition (1)

| # | Melanin (%) | p-value |
|---|---|---|
| (1) Control | 88.44 | — |
| (4) | 101.14 | 0.044 |
| (5) | 110.00 | 0.084 |
| (6) | 68.43 | 0.029 |
| (7) | 81.92 | 0.028 |
| (8) Reference | 104.83 | 0.254 |

The amount of melanin in each of the skin models treated with the test bar soap compositions (4)-(7) were compared to the untreated skin model, and p-values were determined for significance (p-value≤0.05). The results are summarized in Table 8.

TABLE 8
Quantification of Melanin in Skin Model Treated with Test
Bar Soap Compositions (4)-(7) Compared with Untreated skin

| # | Melanin (%) | p-value |
|---|---|---|
| Untreated | 100 | — |
| (4) | 101.14 | 0.439 |
| (5) | 110.00 | 0.054 |
| (6) | 68.43 | 0.005 |
| (7) | 81.92 | 0.051 |
| (8) Reference | 104.83 | 0.257 |

As indicated above, treatment with the control bar soap composition (1) slightly decreased the amount of melanin in the epidermis. However, it was surprisingly and unexpectedly discovered that skin models treated with bar soap composition (5), which included phenylethyl resorcinol alone, significantly increased melanin production in the epidermis. It was also surprisingly and unexpectedly discovered that bar soap composition (4) did not have any significant effect on melanin production in the epidermis compared to the control bar soap composition (1). It was further surprisingly and unexpectedly discovered that bar soap composition (6), including about 0.01 wt % phenylethyl resorcinol and about 0.05 wt % kojic acid (i.e., ratio of 1:5), significantly reduced melanin in the epidermis. This was especially surprisingly since bar soap composition (7), which also included about 0.01 wt % phenylethyl resorcinol and about 0.01 wt % kojic acid (i.e., ratio of 1:1), while demonstrating reduced amount of melanin, did not significantly reduce the amount of melanin as compared to the control bar soap composition (1).

The present disclosure has been described with reference to exemplary implementations. Although a limited number of implementations have been shown and described, it will be appreciated by those skilled in the art that changes may be made in these implementations without departing from the principles and spirit of the preceding detailed description. It is intended that the present disclosure be construed as including all such modifications and alterations insofar as they come within the scope of the appended claims or the equivalents thereof.

What is claimed is:

1. A solid cleansing composition, comprising:
a delivery vehicle; and
one or more whitening agents, wherein the whitening agents comprise one or more of kojic acid, resorcinol, or combinations thereof, and wherein the whitening agents are configured to regulate melanin in or on skin.

2. The solid cleansing composition of claim 1, wherein the kojic acid comprises one or more of 5-Hydroxy-2-(hydroxymethyl)-4H-pyran-4-one, a kojic acid derivative, or combinations thereof.

3. The solid cleansing composition of claim 1, wherein the kojic acid comprises 5-Hydroxy-2-(hydroxymethyl)-4H-pyran-4-one or 5-hydroxy-2-hydroxymethl-γ-pyrone.

4. The solid cleansing composition of claim 1, wherein the kojic acid comprises encapsulated kojic acid.

5. The solid cleansing composition of claim 1, wherein the kojic acid is present in an amount of from about 0.005 wt % to about 2 wt %, based on the total weight of the solid cleansing composition.

6. The solid cleansing composition of claim 1, wherein the resorcinol comprises one or more of benzene-1,3-diol, a derivative thereof, or combinations thereof.

7. The solid cleansing composition of claim 1, wherein the resorcinol comprises phenylethyl resorcinol.

8. The solid cleansing composition of claim 1, wherein the resorcinol is present in an amount of from about 0.005 wt % to about 2 wt %, based on the total weight of the solid cleansing composition.

9. The solid cleansing composition of claim 1, wherein the whitening agents comprise a combination of the kojic acid and the resorcinol.

10. The solid cleansing composition of claim 9, wherein the kojic acid and resorcinol are present in a weight ratio of from about 0.1:1 to about 10:1.

11. The solid cleansing composition of claim 1, wherein the delivery vehicle comprises at least one cleansing component, wherein the cleansing component comprises one or more soaps, one or more synthetic surfactants, or combinations thereof.

12. A method for preparing the solid cleansing composition of claim 1, the method comprising contacting the delivery vehicle and the one or more whitening agents with one another.

13. A method for treating, decreasing, reducing, or preventing hyperpigmentation of skin, the method comprising contacting an effective amount of the solid cleansing composition of claim 1 with the skin.

14. A method for regulating melanin in or on skin, the method comprising contacting an effective amount of the solid cleansing composition of claim 1 with the skin.

15. A method for stabilizing the one or more whitening agents of the solid cleansing composition of claim 1, the method comprising contacting the delivery vehicle and the one or more whitening agents with one another, wherein stabilizing the one or more whitening agents comprises preventing or inhibiting degradation of any of the one or more whitening agents.
